(19) United States
(12) Patent Application Publication
(10) Pub. No.: US 2008/0071274 A1
(43) Pub. Date: Mar. 20, 2008

(54) Percutaneous Screw Assembly and Placement Method

(76) Inventor: Michael D. Ensign, Salt Lake City, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/856,673

(22) Filed: Sep. 17, 2007

Related U.S. Application Data
(60) Provisional application No. 60/844,901, filed on Sep. 15, 2006.
(60) Provisional application No. 60/844,982, filed on Sep. 15, 2006.
(60) Provisional application No. 60/844,987, filed on Sep. 15, 2006.

(51) Int. Cl. A61B 17/58; A61B 17/56

(52) U.S. Cl. 606/61; 606/103; 606/73

(57) ABSTRACT

A connection member for percutaneously coupling to one or more orthopedic fasteners includes a fastener head securing member including a fastener head securing orifice having an axis defined by a wall member terminating in a seating member, an adjustable compression member coupled to a surface of the wall member, a rod coupled to a lower half of the wall member, and a fastener head receiving orifice formed in the wall member, wherein the fastener head receiving orifice is formed transverse to and intersects the screw head securing orifice axis.

### RELATED APPLICATIONS

This application claims the benefit under 35 U.S.C. §119(e) of U.S. Provisional Patent Application Nos. 60/844,901; 60/844,982; and 60/844,987 all filed Sep. 15, 2006 titled “Percutaneous Screw Assembly”; “Low Profile Percutaneous Screw Design” and “System and Method for a Percutaneous Placement of a Rod System” respectively, which applications are incorporated herein by reference in their entireties.

### TECHNICAL FIELD

The present exemplary system and method relates to medical devices. More particularly, the present exemplary system and method relates to orthopedic rod placement devices.

### BACKGROUND

The use of bone stabilization/fixation devices to align or position bones is well established. Furthermore, the use of spinal bone stabilization/fixation devices to align or position specific vertebrae or a region of the spine is well established. Typically such devices for the spine utilize a spinal fixation element, comprised of a relatively rigid member such as a plate or rod that is used as a coupler between adjacent vertebrae. Such a spinal fixation element can effect a rigid positioning of adjacent vertebrae when attached to the pedicle portion of the vertebrae using pedicle bone anchorage screws. Once the coupled vertebrae are spatially fixed in position, procedures can be performed, healing can proceed, or spinal fusion may take place.

Spinal fixation elements may be introduced to stabilize the various vertebrae of the spine. Some devices for this purpose are designed to be attached directly to the spine, but the generally invasive nature of standard paraspinal approach used to implant these devices may pose drawbacks. For example, muscle disruption and blood loss may result from standard paraspinal implantation approaches.

### SUMMARY

In one of many possible embodiments, the present exemplary system provides a connection member for coupling to one or more pedicle screws including a tulip member having a screw head securing orifice defined by a wall member terminating in a seating member, a set screw member coupled to a surface of the wall member, a rod coupled to the wall member, and a pedicle screw head receiving orifice formed in the wall member, wherein the pedicle screw head receiving orifice is formed transverse to and intersects the screw head securing orifice.

Another embodiment of the present exemplary system and method provides a method for coupling a connection member to a pedicle screw including inserting a head of a pedicle screw through a first orifice in the connection member along a first line of motion, orienting the connection member with respect to the pedicle screw such that the screw shaft is oriented perpendicular to the first line of motion, seating the screw head in the connection member, and securing the position of the pedicle screw in the connection member.

According to yet another exemplary embodiment of the present exemplary system and method, a coupling and connection member includes a tulip member having a screw head securing orifice defined by a wall member terminating in a seating member, a set screw member coupled to a surface of the wall member, a rod coupled to the lower half of the wall member, and a pedicle screw head receiving orifice formed in the wall member, wherein the pedicle screw head receiving orifice is formed transverse to and intersects the screw head securing orifice. According to one exemplary embodiment, a screw driving element may be formed in the wall member adjacent to the rod.

According to yet another exemplary embodiment of the present exemplary system and method, a coupling and connection member includes a tulip member having an orifice defined therein for receiving a guide wire.

### BRIEF DESCRIPTION OF THE DRAWINGS

The accompanying drawings illustrate various embodiments of the present system and method and are a part of the specification. The illustrated embodiments are merely examples of the present system and method and do not limit the scope thereof.

FIG. 1 is a perspective view of a percutaneous connection member, according to one exemplary embodiment.

FIGS. 2A, 2B, 2C, and 2D are respectively front, top, side cross-sectional, and bottom views of the percutaneous connection member of FIG. 1, according to a number of exemplary embodiments.

FIGS. 3A through 3D illustrate a tulip first placement method, according to one exemplary embodiment.

FIG. 4 illustrates the steps of a tulip first placement method, according to one exemplary embodiment.

FIGS. 5A through 5C illustrate a rod first placement method, according to one exemplary embodiment.

FIG. 6 illustrates the steps of a rod first placement method, according to one exemplary embodiment.

FIGS. 7A through 9B illustrate the mechanics of engaging the exemplary percutaneous connection member illustrated in FIG. 1 on the head of a pedicle screw, according to one exemplary embodiment.

FIG. 10 illustrates a perspective view of a percutaneous connection member, according to one alternative embodiment.

FIG. 11 is a side view illustrating the coupling of a rod to the lower half of a tulip housing member, according to one exemplary embodiment.

FIGS. 12A through 12E illustrate various stages of placement and fixation of a percutaneous connection member, according to one exemplary embodiment.

FIGS. 13A through 13C illustrate a bottom perspective, a top perspective, a bottom, and a side assembled view of a percutaneous connection member, according to one exemplary embodiment.

FIGS. 14A through 14E illustrate the placement of a percutaneous connection member using an exemplary placement tool, according to one exemplary embodiment.

Throughout the drawings, identical reference numbers designate similar, but not necessarily identical, elements.

### DETAILED DESCRIPTION

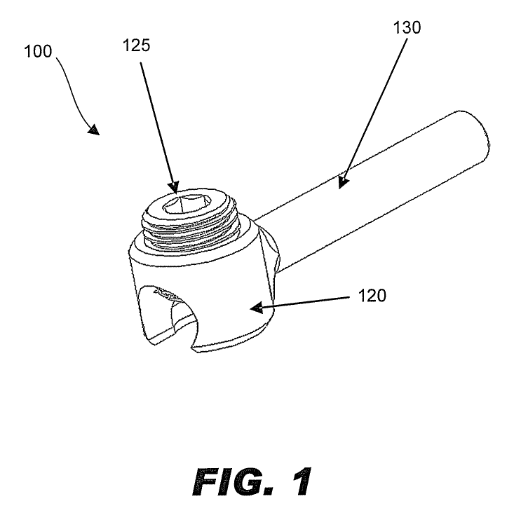

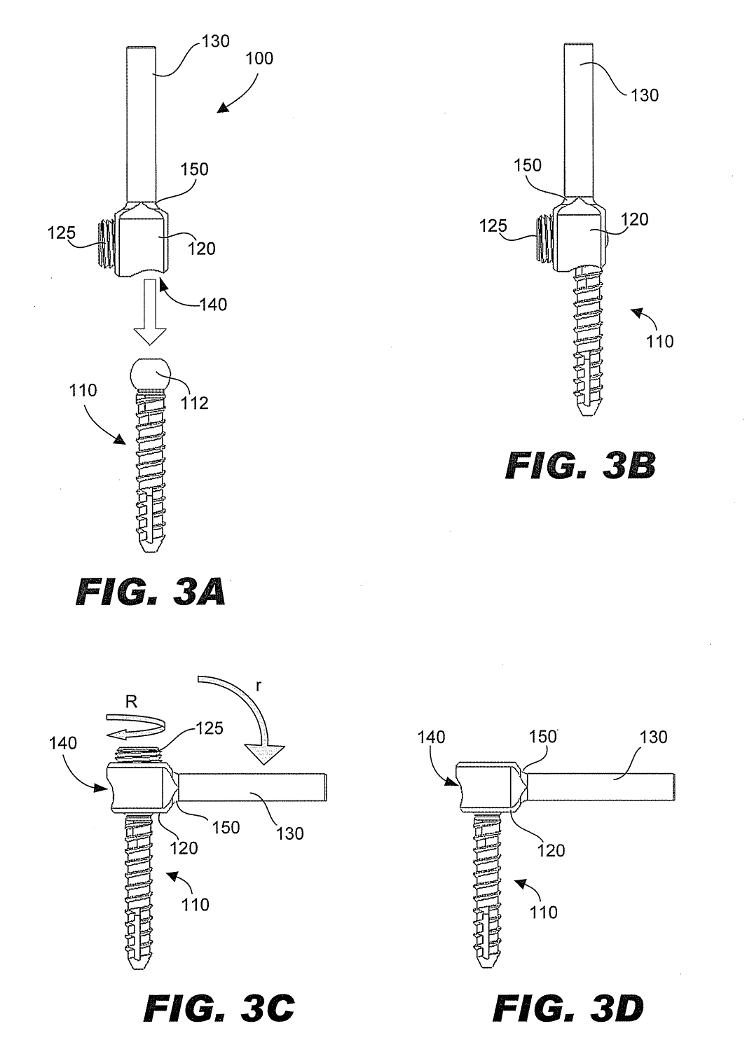

The present exemplary system and methods, illustrated by FIGS. 1 through 14E provide a connection member that can be used for any number of orthopedic rod placement systems. According to the present exemplary system and method, percutaneous screw placement is facilitated. Specifically, the present exemplary systems and methods provide for placement of the pedicle screws first, followed by easy placement of the rod and one or more tulips simultaneously. As illustrated in FIGS. 3A through 6, the present exemplary connection member may be percutaneously inserted either rod first, or tulip first. Furthermore, due to the fixed connection between the rod and the tulip of the present exemplary system configuration, the profile and volume of the present exemplary system are reduced, compared to traditional systems.

The present exemplary system and method are elegant solutions to maintaining polyaxial movement in the orthopedic rod placement system. Additionally, according to one exemplary embodiment, the illustrated connection member may be cannulated.

By way of example, pedicle screw systems may be fixed in the spine in a posterior lumbar fusion process via minimally invasive surgery (MIS) techniques. The systems are inserted into the pedicles of the spine and then interconnected with rods to manipulate (e.g., correct the curvature, compress or expand, and/or structurally reinforce) at least portions of the spine. Using the MIS approach to spinal fixation and/or correction surgery has been shown to decrease a patient's recovery time and reduce the risks of follow-up surgeries.

Traditional percutaneous fixation techniques are really only percutaneous in name. That is, they still require significant paraspinous tissue damage in order to fixedly couple a connector rod between two or more tulips. This is due in part to the implants that are available to the surgeon. The present exemplary system and method allows a surgeon to place spinal screws and rods via a true percutaneous approach by providing for pivoting of the rod beneath the skin in a fascial plane, lateral to the multifidous.

The ability to efficiently perform spinal fixation and/or correction surgeries using MIS techniques is enhanced by the use of pedicle screw systems provided in accordance with the present exemplary systems and methods, which systems and methods provide a number of advantages over conventional systems. For example, a pedicle screw system in accordance with one embodiment of the present exemplary system and method provides the advantage that the pedicle screw may be inserted into the bone without being pre-operatively coupled with the rod-coupling assembly (hereinafter referred to as a tulip assembly). This is advantageous because the surgeon often needs to do other inter-body work after inserting the pedicle screw, but before attaching the larger and bulkier tulip assembly. Such an advantageous pedicle screw system may be even more crucial when using MIS techniques because the inter-body spatial boundaries in which the surgeon must work may be quite limited.

The term “distraction,” when used herein and when used in a medical sense, generally relates to joint surfaces and suggests that the joint surfaces move perpendicular to one another. However when “traction” and/or “distraction” is performed, for example on spinal sections, the spinal sections may move relative to one another through a combination of distraction and gliding, and/or other degrees of freedom.

Furthermore, the terms “percutaneous pedicle screw system” and “connection member” will be used interchangeably herein to refer to a structure that includes a tulip housing configured to capture and securely receive a head portion of a pedicle screw, and includes a rod member directly coupled to the outer wall of the tulip housing.

In the following description, certain specific details are set forth in order to provide a thorough understanding of various embodiments of the present percutaneous pedicle screw system. However, one skilled in the relevant art will recognize that the present exemplary system and method may be practiced without one or more of these specific details, or with other methods, components, materials, etc. In other instances, well-known structures associated with pedicle screws have not been shown or described in detail to avoid unnecessarily obscuring descriptions of the embodiments of the systems and methods.

Unless the context requires otherwise, throughout the specification and claims which follow, the word “comprise” and variations thereof, such as, “comprises” and “comprising” are to be construed in an open, inclusive sense, that is as “including, but not limited to.”

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

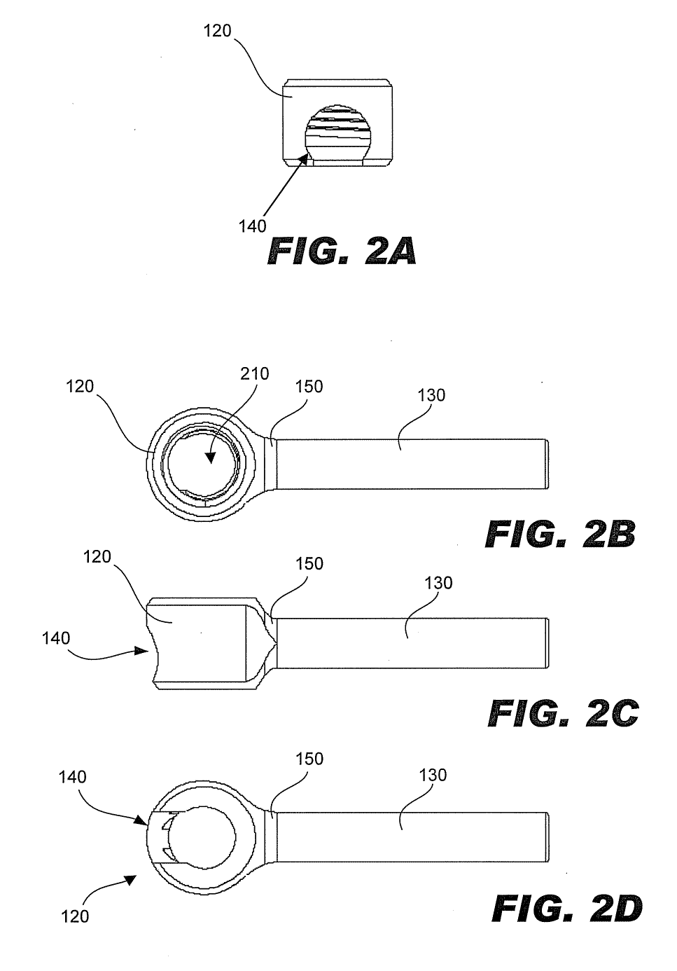

FIG. 1 illustrates a perspective view of a percutaneous screw assembly system (100), according to one exemplary embodiment. As illustrated in FIG. 1, the main tulip housing (120) of the screw assembly system (100) is configured to be coupled to the top portion of a pedicle screw (not shown). Additionally, as illustrated in FIG. 1, a connector rod (130) is directly coupled to the outer wall of the main tulip housing (120) via a rod coupling feature (150). Further, a screw head reception orifice (140) is formed in the side wall of the main tulip housing (120), and a center thru bore (210) allowing portions of the pedicle screw (1 10) to pass there through. Further details of the present exemplary percutaneous screw assembly system (100) are shown in FIG. 2.

In addition to the exemplary pedicle screw (110), the exemplary percutaneous pedicle screw system (100) includes a tulip assembly including a main tulip housing (120) permanently coupled to the connector rod (130) by a rod coupling feature (150). Additionally, the tulip housing (120) includes a number of features that facilitate reception, rotation, and coupling of a head portion (112) of a pedicle screw (110), according to one exemplary embodiment. As shown in FIG. 1, a set screw (125) configured to rotatably advance as it is engaged with internal threads formed in the main tulip housing is configured to secure a head portion (112) of a pedicle screw (110), according to one exemplary embodiment. Additionally, as illustrated in FIG. 2A, the exemplary tulip housing (120) includes a head reception orifice (140) formed in a sidewall of the main tulip housing (120). As shown, the tulip housing (120) includes an inner thru bore (210) that extends concentrically along the axis of the cylindrically shaped tulip housing, beginning at a threaded portion and exiting at the bottom of the tulip housing (120) past the head reception orifice (140). According to one exemplary embodiment, the diameter of the screw head reception orifice (140) is sufficient to receive the pedicle screw head. However, the exiting orifice of the inner thru bore (210) adjacent to the head reception orifice (140) is sized such that the head portion (112) of a pedicle screw (110) is unable to pass there through, while the threaded shaft portion is able to pass.

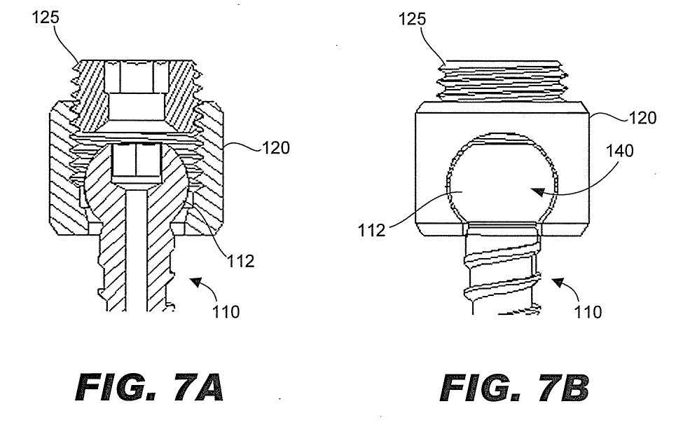

Additionally, according to one exemplary embodiment, a number of internal features and components, described below with reference to FIGS. 7A through 9B, are positioned within the inner bore (210) to receive and couple the head portion (112) of the pedicle screw (110) when the set screw (125) is engaged. According to the present exemplary embodiment, the internal features and the screw head reception orifice (140) facilitate percutaneous reception and locking of a pedicle screw head (112). Additionally, as illustrated in FIG. 1, a set screw (125) or other fastener is inserted in the top of the inner bore (210). According to one exemplary embodiment, the set screw (125) is advanced along the threads formed on the inner bore to further engage the internal features with the pedicle screw head (112), thereby forming a secure positional lock.

As mentioned, the exemplary percutaneous pedicle screw system (100) includes the connector rod (130) securely coupled to the side wall of the tulip housing (120) by a rod coupling feature (150). According to one exemplary embodiment, the connector rod (130) may be coupled to the side wall of the tulip housing (120) using any number of joining methods known in the art including, but in no way limited to, welding, brazing, or the use of adhesives. Alternatively, the rod coupling feature (150) may include any number of mechanical joining features including, but in no way limited to, a threaded engagement feature or an interference press fit feature.

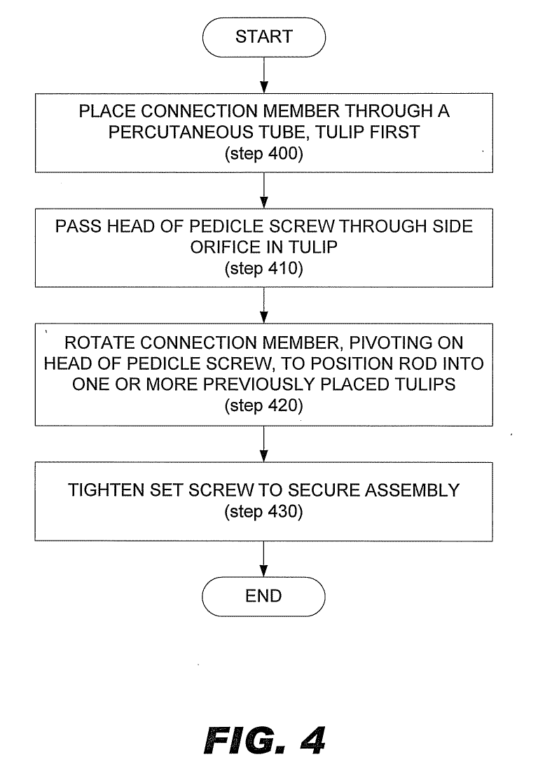

With the exemplary percutaneous pedicle screw system (100) illustrated above, a number of percutaneous connection member placement methods may be performed. By way of example only, FIGS. 3A through 3D illustrate a tulip first placement method that is facilitated by the present exemplary pedicle screw system, according to one exemplary embodiment. The exemplary tulip placement method is detailed in FIG. 4. As described, the exemplary tulip first placement method begins by first placing a number of pedicle screws (110) in desired patient locations and passing a connection member through a percutaneous tube, tulip first (step 400; FIG. 4). As illustrated in FIG. 3A, the percutaneous pedicle screw system (100) may be placed through a percutaneous tube (not shown) such that the screw head receiving orifice (140) leads the pedicle screw system (100) down the tube and initially engages the head portion (112) of the pedicle screw (110). Once in the proper location, the head of the pedicle screw (110) may be passed through the screw head receiving orifice (step 410; FIG. 4), as illustrated in FIG. 3B. As mentioned previously, the screw head receiving orifice (140) is sized such that the had portion (112) of a pedicle screw (110) easily passes through the orifice.

When the percutaneous pedicle screw system (100) is seated on the head portion (112) of the pedicle screw (110), the screw system (100) is rotated (r), pivoting on the head portion of the pedicle screw, to position the rod (130) onto one or more previously placed tulips (step 420). According to this exemplary embodiment, the lower portion of the pedicle screw (110) is able to pass between a gap created by the screw head receiving orifice (140) and the central thru bore (210). When the percutaneous pedicle screw system (100) is correctly placed, the set screw (125) is rotated (R) to advance the set screw onto the head portion (112) of the pedicle screw (110), thereby locking the pedicle screw system (100) into place and assuring assembly (step 430). As illustrated in FIG. 3D, once the set screw (125) is advanced, the assembly remains securely coupled to the head portion (112) of the pedicle screw (110).

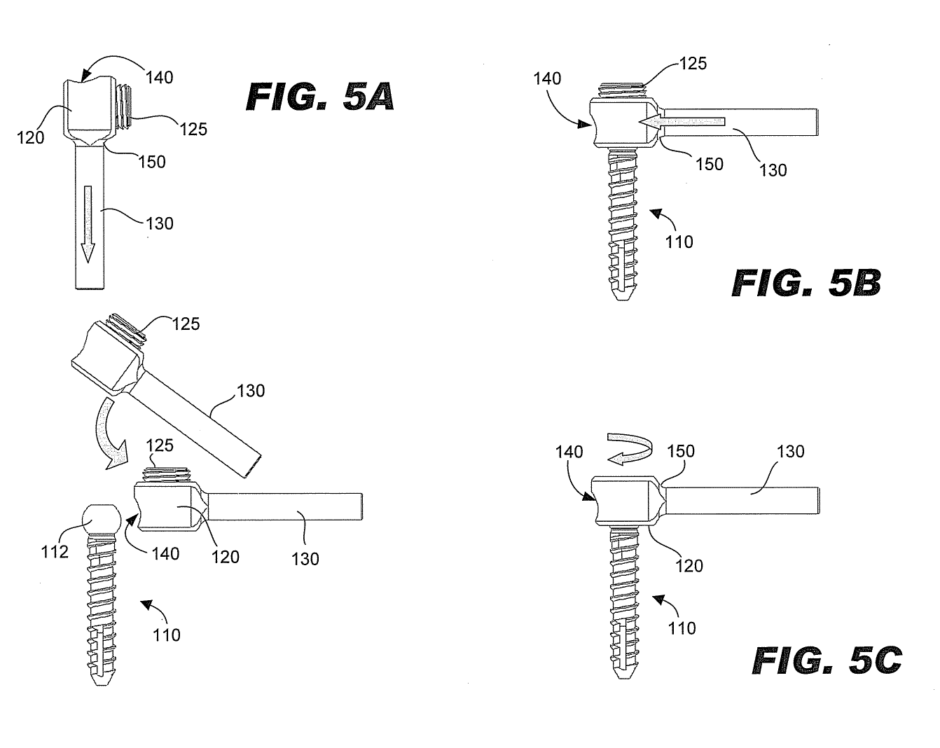

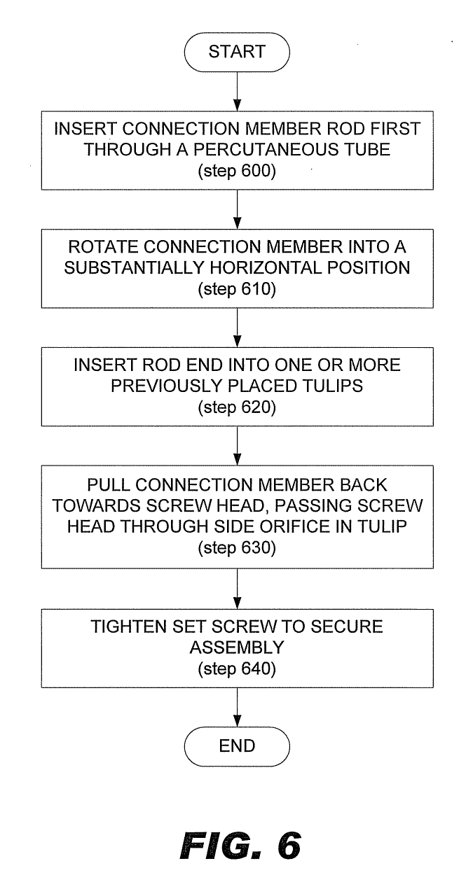

Alternatively, the present exemplary pedicle screw system (100) may be placed in a desired location by a rod first placement method, as illustrated in FIGS. 5A through FIG. 6. As illustrated in FIG. 5A, the exemplary rod first placement method begins by first placing a number of pedicle screws (110) in desired patient locations and passing a connection member through a percutaneous tube, rod first (step 600; FIG. 6). As illustrated in FIG. 5A, the percutaneous pedicle screw system (100) may be placed through a percutaneous tube (not shown) such that the rod (130) leads the pedicle screw system (100) down the tube. As the rod (130) nears the head portion (112), the pedicle screw system (100) is rotated into a substantially horizontal position (step 610; FIG. 6). As illustrated in FIG. 5A, the screw head receiving orifice (140) is positioned adjacent to the head portion (112) of the desired pedicle screw (110).

Continuing with the method of FIG. 6, once the present exemplary pedicle screw system (100) is correctly positioned, the rod end (130) of the system is inserted into one or more previously placed tulips (step 620; FIG. 6). After the rod (130) is captured in one or more previously placed tulips, the percutaneous pedicle screw system (100) is pulled back (F) towards the head portion (112) of the pedicle screw (110), passing the screw head portion (112) through the screw head receiving orifice (140) into the central thru bore (step 630; FIG. 6). As illustrated in FIG. 5C, once the tulip housing (120) correctly contains the head portion (112) of the pedicle screw (110), the set screw (125) is tightened to lock the percutaneous pedicle screw system (100) to the head portion of the pedicle screw (110), thereby securing the assembly (step 640; FIG. 6).

As illustrated in FIGS. 3A through 6, both assembly methods are completed by advancing the exemplary set screw (125), thereby securing the pedicle screw system (100) to the head portion (112) of the exemplary pedicle screw (110). FIGS. 7A through 9B illustrate the interaction between the exemplary percutaneous pedicle screw system (100) and the head portion (112) of the exemplary pedicle screw (110) as the set screw (125) is advanced. As shown in FIGS. 7A and 7B, the head portion (112) of the exemplary pedicle screw (110) is passed through the screw head receiving orifice (140) as the set screw (125) is fully retracted. This allows for clear passage of the head portion (112) of the pedicle screw (110) through the screw head receiving orifice (140).

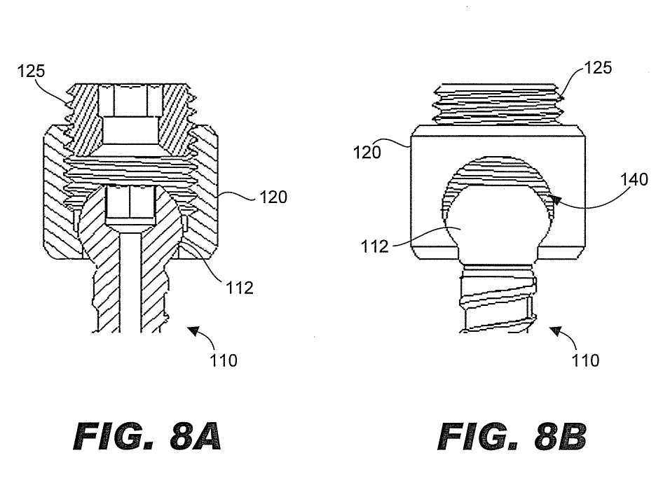

When the head portion (112) of the exemplary pedicle screw (110) is fully entered into the central thru bore (210) of the tulip housing (120), the screw head is seated in a spherical bore formed on the lower portion of the central thru bore in line with the axis of the set screw (125). As illustrated in FIGS. 8A and 8B, when the screw head is seated, the head portion (112) of the pedicle screw is at least partially retained in the central thru bore (210) as the head portion (112) is no longer lined up with the axis of the screw head receiving orifice (140).

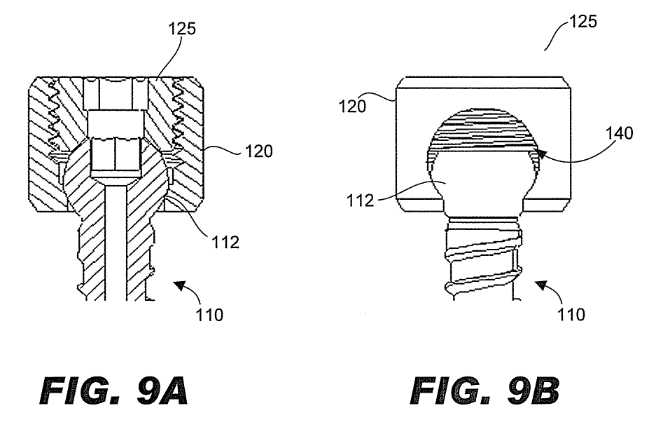

Once seated, the set screw (125) is advanced until it contacts and secures the screw head (112) into the central thru bore (210), as illustrated in FIGS. 9A and 9B. Placement of the set screw (125) over the head portion (112) of the pedicle screw (110) prevents the head portion from rising and being forced out of the screw head receiving orifice.

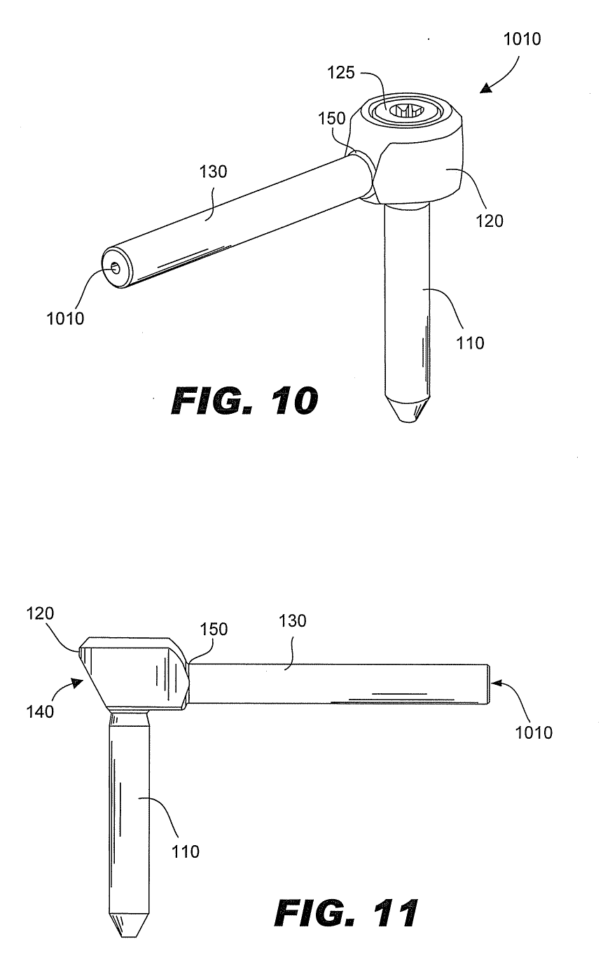

As illustrated in FIGS. 3A through 6, both assembly methods rely on substantially precise placement of the present exemplary pedicle screw system (100) over the head portion (112) of the pedicle screw (110). Consequently, the present exemplary system and method provides a plurality of designs configured to aid in the precise placement of the present exemplary pedicle screw system (100). FIGS. 10-12E illustrate an exemplary feature configured to aid in the placement of the present exemplary pedicle screw system, according to one exemplary embodiment. As is well known in the art, the use of a guide wire, such as a Kischner or K-wire facilitates precise placement of instruments and implants. Consequently, the exemplary pedicle screw system (1000) illustrated in FIG. 10 facilitates reception of a K-wire through a cannulated rod (130) having a guide wire lumen (1010) formed therein. Specifically, as illustrated in FIG. 11, the first exemplary configuration includes a tulip housing (120) with a cannulated rod (130) formed thereon. As illustrated in FIG. 11, the cannulated rod (130) including a guide wire lumen (1010) is positioned on the lower half of the tulip housing (120) to concentrically align the guide wire lumen with the axis of the screw head receiving orifice (140). By moving the cannulated rod (130) to the lower half of the tulip housing (120), a number of desired features are achieved. First, the lower portion of the tulip housing (120) which receives a majority of the stresses when the set screw is advanced to the head portion (112) of the pedicle screw (110). The coupling of the cannulated rod (130) to the tulip housing (120) on the lower portion of the tulip housing (120) strengthens the lower region of the tulip housing. Additionally, the placement of the cannulated rod (130) on the lower half of the tulip housing (120) aligns the guide wire lumen (1010) with the axis of the screw head receiving orifice (140).

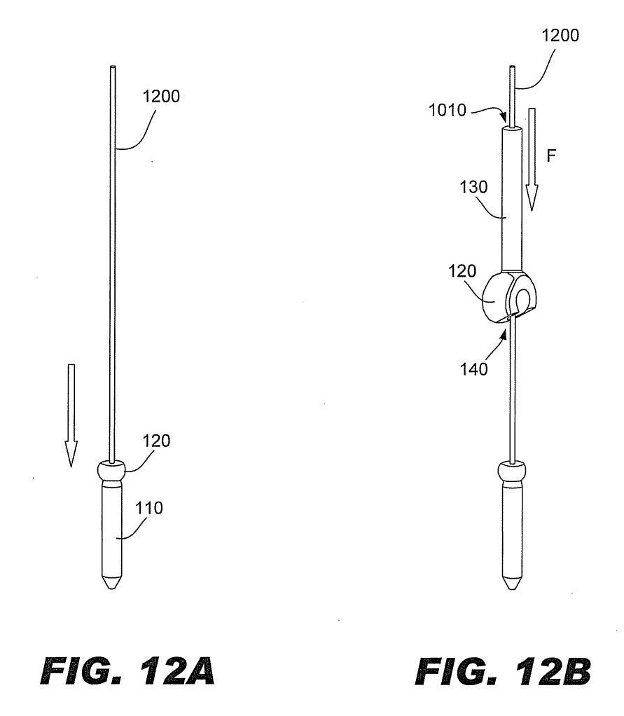

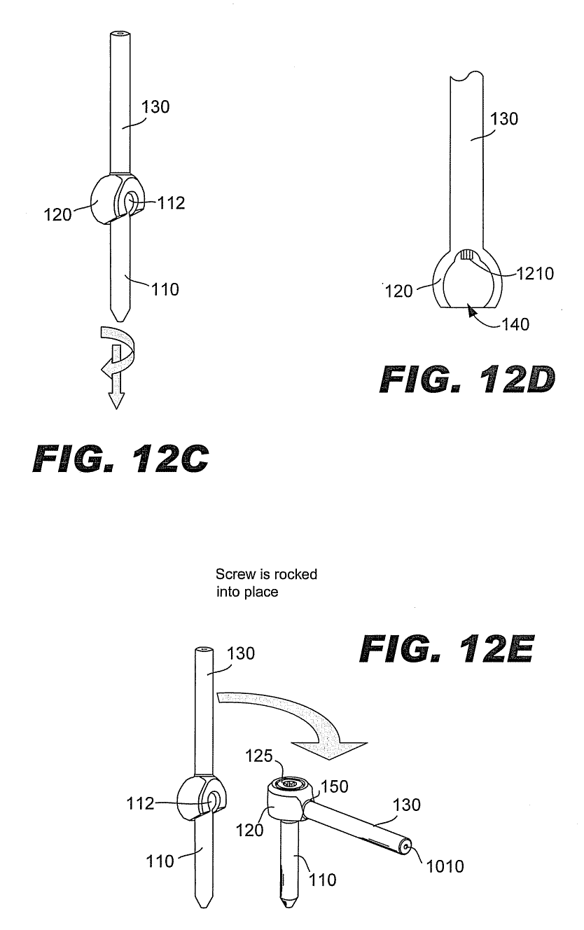

FIGS. 12A and 12B illustrate how the present exemplary rod placement facilitates the precise guidance of the exemplary pedicle screw system (1000). As illustrated in FIG. 12A, the K-wire (1200) is first placed onto the head portion (112) of the desired pedicle screw (110). Once properly placed, the K-wire may be passed through the cannulated pedicle screw system (1000), thereby mating the head receiving orifice (140) with the head portion (112) of the pedicle screw (110).

Furthermore, the placement of the cannulated rod (130) on the outer wall of the tulip housing (120) allows for the internal portion of the exemplary pedicle screw system (1000) to include a number of useful features. For example, as illustrated in FIGS. 12C and 12D, a driver feature (1210) for driving the pedicle screws (110) is formed in the inner surface of the tulip housing. According to one exemplary embodiment, the male driver feature (1210) is cannulated and is concentric with the cannula of the cannulated rod. Consequently, placement of the exemplary pedicle screw system over the K-wire (1200) guides the driver feature directly to the female driving feature (not shown) of the head portion of the pedicle screw (110). As illustrated in FIG. 12C, once engaged, the male driver feature may be used to drive the pedicle screw (illustrated by arrows). When the exemplary pedicle screw (110) is properly placed, the exemplary pedicle screw system (1000) is retracted from the female driving feature and rocked (r) into place, as shown in FIG. 12E.

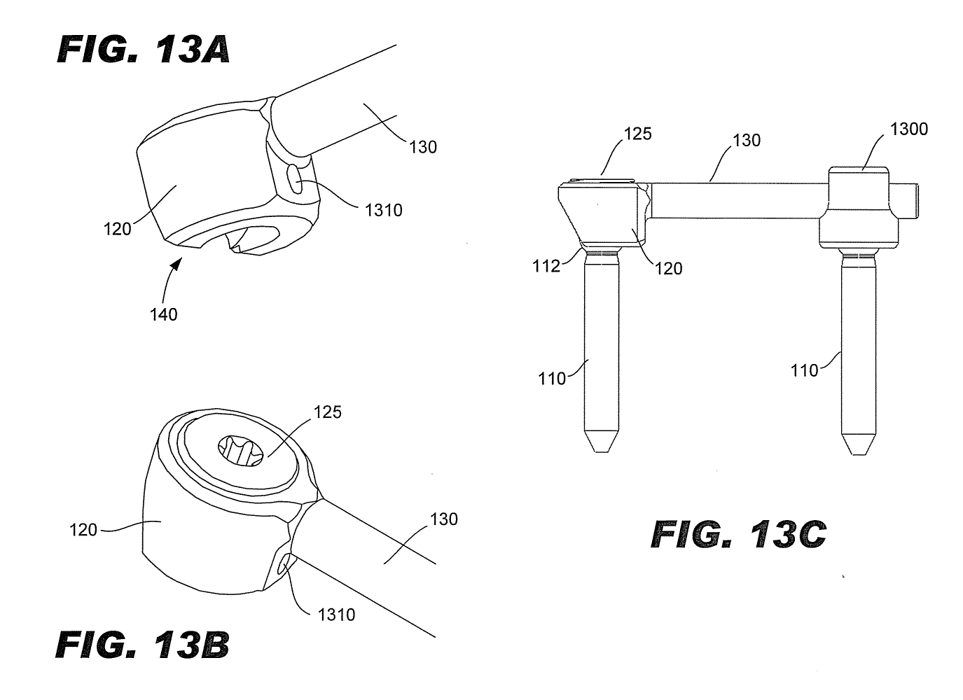

Alternatively, the rod member (130) of the exemplary pedicle screw system (1300) may be raised to the top portion of the tulip housing (120), as illustrated in FIGS. 13A through 13C. According to this exemplary embodiment, the top surface of the rod member (130) is positioned such that it is substantially even with the top surface of the tulip housing (120). According to this exemplary embodiment, a guide wire orifice (1310) can be formed in the wall of the tulip housing, as illustrated in FIGS. 13A and 13B. According to one exemplary embodiment, the guide wire orifice (1310) is placed in the wall of the tulip housing (120) such that the guide wire orifice is substantially concentric with the screw head receiving orifice (140).

Furthermore, as illustrated in FIG. 13C, the placement of the rod member (130) adjacent to the top surface of the tulip housing (120) reduces the profile of the resulting structure. As shown, the rod member (130) extends to another pedicle screw configuration (1320). By rising the rod member (130) to the top of the tulip housing (120), other height adding components and tulip housing itself are reduced to a minimum.

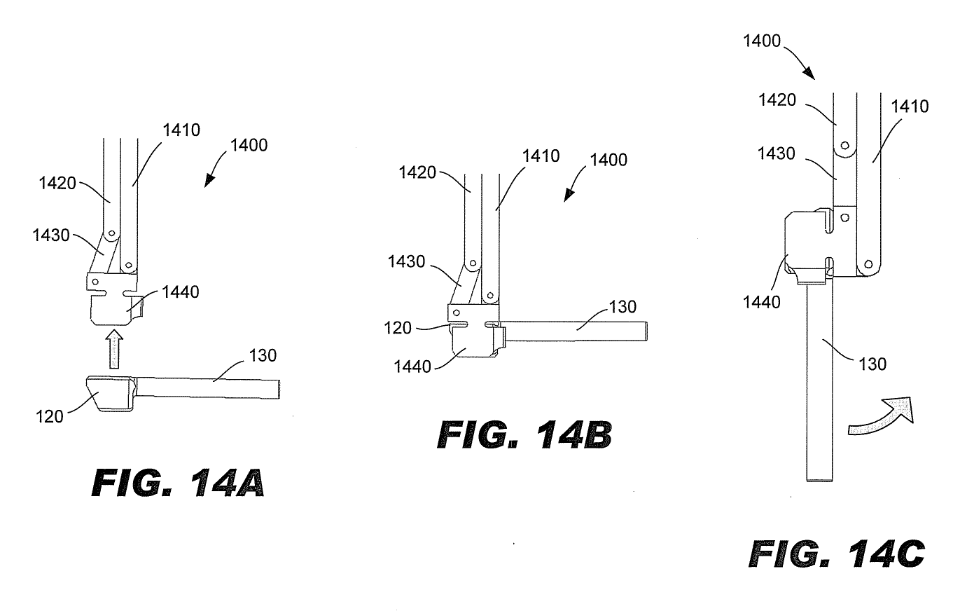

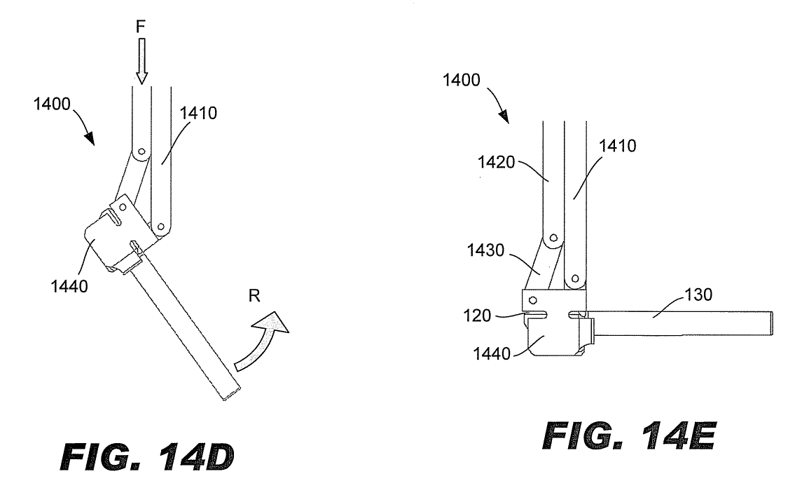

Throughout the present exemplary specification, a number of pedicle screw placement systems and methods have been described. Correct placement of the present exemplary pedicle screw systems requires an instrument capable of grasping and pivoting the pedicle screw placement system. FIGS. 14A through 14E illustrate an exemplary instrument for manipulating the present exemplary pedicle screw systems.

As illustrated in FIG. 14A, the exemplary connection member instrument (1400) includes a kinematically defined four-bar mechanism including a slider, configured to selectively rotate the pedicle screw systems with a single actuation. As illustrated in FIG. 14A, the exemplary connection member instrument (1400) includes a stationary member (1410), an actuator member (1420) coupled to an input generator (not shown), a linking member (1430), and a housing capture member (1440). As illustrated, the housing capture member (1440) is shaped and includes a number of features configured to couple a tulip housing (120).

As illustrated in FIG. 14A, manipulation of an exemplary tulip housing (120) begins by first forcing the tulip housing (120) into the housing capture member (1440). According to one exemplary embodiment, the housing capture member (1440) may be configured to couple the exemplary tulip housing (120) by a compression fit, engagable mating features, threads, and the like. FIG. 14B illustrates a fully engaged tulip housing (120) according to one exemplary embodiment. Alternatively, the housing capture member (1440) may be configured to capture the rod member (130) rather than the tulip housing.

Once the desired tulip housing (120) is engaged in the housing capture member, the coupled tulip housing may be selectively oriented by manipulation of the actuator member (1420). As shown in FIGS. 14C, 14D, and 14E, actuation of an input generator, such as a trigger, a clamp, pituitary ronguers, or a pistol grip slideably moves the actuator member (1420) parallel with the stationary member (1410). As the actuator member (1420) proceeds, the pin joining the linking member and the actuator member (1420) remains parallel with the stationary member (1410) while the portion of the linking arm (1430) that is coupled to the housing capture member (1440), also referred to as an engagement collar, is forced forward, and out away from the stationary bar (1410). Consequently, the housing capture member (1440) is rotated (R) between zero and 90 degrees, as shown in FIG. 14E. Once in a desired horizontal position, the exemplary connection member instrument (1400) can be used to join the tulip housing (120) to a head portion of a pedicle screw.

A number of embodiments of the present exemplary system and method have been described and are illustrated in the accompanying figures. Nevertheless, it will be understood that various modifications may be made without departing from the spirit and scope of the present exemplary systems and methods. For example, while the exemplary implementations have been described and shown using screws to anchor into bony structures, the scope of the present exemplary system and methods is not so limited. Any means of anchoring can be used, such as a cam, screw, staple, nail, pin, or hook.

The preceding description has been presented only to illustrate and describe embodiments of invention. It is not intended to be exhaustive or to limit the invention to any precise form disclosed. Many modifications and variations are possible in light of the above teaching. It is intended that the scope of the invention be defined by the following claims.

What is claimed is:

1. A connection member for percutaneously coupling to one or more orthopedic fasteners comprising:
a fastener head securing member including a fastener head securing orifice having an axis defined by a wall member terminating in a seating member;
an adjustable compression member coupled to a surface of said wall member;
a rod coupled to said wall member; and
a fastener head receiving orifice defined in said wall member, wherein said fastener head receiving orifice is formed transverse to and intersects said screw head securing orifice axis.

2. The connection member of claim 1, wherein said rod comprises a cannulated rod.

3. The connection member of claim 1, wherein said rod is coupled to a lower half of said wall member.

4. The connection member of claim 2, wherein said fastener head securing member further defines an engagement recess defined in said wall member adjacent to said rod, wherein said engagement recess is defined in said wall member opposite said fastener head receiving orifice.

5. The connection member of claim 4, further comprising a screw driving member formed in said engagement recess.

6. The connection member of claim 1, wherein said rod is coupled adjacent to a top surface of said wall member.

7. The connection member of claim 1, further comprising a guide wire orifice defined in said wall member.

8. A method for coupling a connection member having a tulip coupled to a rod to at least one orthopedic fastener having a fastening shaft, comprising:
passing a head of said orthopedic fastener through a first orifice in said connection member along a first line of motion;
engaging said head of said orthopedic fastener with a fastener driving member in an engagement recess in said tulip;
driving said orthopedic fastener with said tulip;
withdrawing said head of said orthopedic fastener from said engagement recess;
orienting said connection member with respect to said orthopedic fastener such that said fastening shaft is oriented substantially perpendicular to said first line of motion;
seating said orthopedic fastener head in said connection member; and
positionally fixing said orthopedic fastener in said connection member.

9. The method of claim 8, wherein said method further comprises:
passing said orthopedic fastener over a k-wire to a desired orthopedic location; and
passing said orthopedic fastener over said k-wire.

10. The method of claim 8, wherein said coupling of said connection member to said at least one orthopedic fasteners is performed percutaneously.

11. The method of claim 10, wherein said passing a head of said orthopedic fastener through a first orifice comprises passing said connection member through a percutaneous tube, tulip first.

12. The method of claim 10, wherein said passing a head of said orthopedic fastener through a first orifice comprises passing said connection member through a percutaneous tube, rod first.

13. An orthopedic fixation system, comprising:
a first connection member including:
a fastener head securing member including a fastener head securing orifice having an axis defined by a wall member terminating in a seating member;
an adjustable compression member coupled to a surface of said wall member;
a rod coupled to an upper half of said wall member; and
a fastener head receiving orifice defined in said wall member, wherein said fastener head receiving orifice is formed transverse to and intersects said screw head securing orifice axis; and
a second connection member including:
an inner member including a lower portion configured to couple said head of said bone fixation device and an upper portion including a plurality of flexible protrusions extending from said lower portion, said plurality of flexible protrusions being configured to provisionally couple a rod;
an outer member configured to house said inner member; and
a cap including a generally planar top and at least one locking member extending from said top, said at least one locking member configured to compress said flexible protrusions to securely lock said rod;
wherein said cap is configured to be seated within said outer member when fully engaged with said inner member.

14. The orthopedic fixation system of claim 13, wherein said first connection member further comprises a k-wire hole defined in a lower half of said wall member.

15. An implant positioning device, comprising:
an actuator member; and
an engagement collar coupled to said actuator member such that an actuation of said actuator member rotates said engagement collar;
wherein said engagement collar is configured to selectively engage a connection member.

16. The implant positioning device of claim 15, wherein said actuator member is configured to rotate said engagement collar approximately 90 degrees.

17. The implant positioning device of claim 15, wherein said actuator member comprises pituitary ronguers.

18. The implant positioning device of claim 15, wherein said actuator member is configured to be selectively actuated to position said friction collar at any orientation between 0 and approximately 90 degrees.

19. The implant positioning device of claim 15, wherein said engagement collar is configured to be slideably released from an engaged tulip.

20. The implant positioning device of claim 15, wherein said engagement collar is configured to threadably or frictionally engage said tulip.

21. The implant positioning device of claim 15, wherein said engagement collar is configured to selectively engage a rod.
